(19) United States
(12) Patent Application Publication
(10) Pub. No.: US 2008/0262474 A1
(43) Pub. Date: Oct. 23, 2008

(54) MEDICAL DEVICE

(75) Inventor: Clay W. Northrop, Salt Lake City, UT (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/738,125

(22) Filed: Apr. 20, 2007

(51) Int. Cl. A61M 25/098

(52) U.S. Cl. 604/529

(57) ABSTRACT

Medical devices and methods for making and using the same. An example medical device may include an elongate shaft having a proximal region and a distal region. A tubular member may be disposed over the distal region of the shaft. The tubular member may have an outer surface and may have a plurality of slots formed therein. A coil may be disposed adjacent the tubular member.

### FIELD OF THE INVENTION

The present invention pertains to intracorporal medical devices, for example, intravascular guidewires, catheters, and the like as well as improved methods for manufacturing and using such medical devices. More particularly, the invention relates to medical devices including an elongate tubular member having a plurality of slots formed therein, and a coil member disposed about the tubular member.

### BACKGROUND

A wide variety of intracorporeal medical devices have been developed for medical use, for example, intravascular use. Some of these devices include guidewires, catheters, and the like. Of the known medical devices, each has certain advantages and disadvantages. There is an ongoing need to provide alternative medical devices as well as alternative methods for manufacturing and using medical devices.

### BRIEF SUMMARY

The invention provides design, material, and manufacturing method alternatives for intracorporal medical devices. An example medical device includes a tubular member having a plurality of slots formed therein. A coil may be disposed adjacent the tubular member. Some of these and other features and characteristics of the inventive devices and methods are described in more detail below.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view of an example medical device disposed in a blood vessel;

FIG. 2 is a partial cross-sectional side view of an example medical device;

FIG. 2A is a partial cross-sectional side view of another example medical device;

FIG. 2B is a partial cross-sectional side view of another example medical device;

FIG. 3 is a partial cross-sectional side view of another example medical device;

FIG. 4 is a partial cross-sectional side view of another example medical device;

FIG. 5 is a partial cross-sectional side view of another example medical device; and

FIG. 6 is a partial cross-sectional side view of another example medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

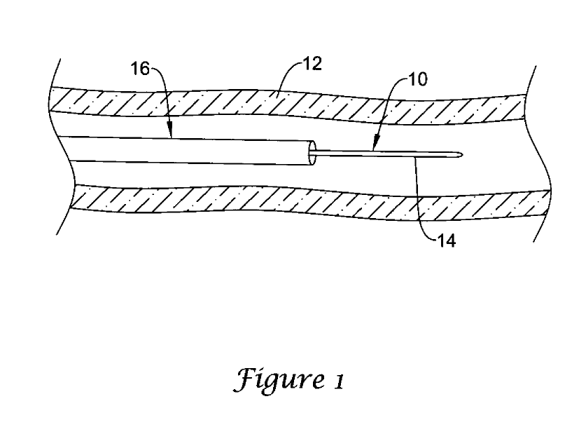

FIG. 1 is a plan view of an example medical device 10, for example a guidewire, disposed in a blood vessel 12. Guidewire 10 may include a distal section 14 that may be, as is well known in the art, generally configured for use within the anatomy of a patient. Guidewire 10 may be used for intravascular procedures according to common practice and procedure. For example, guidewire 10 may be used in conjunction with another medical device 16, which may take the form of a catheter, to treat and/or diagnose a medical condition. Of course, numerous other uses are known amongst clinicians for guidewires and other similarly configured medical devices.

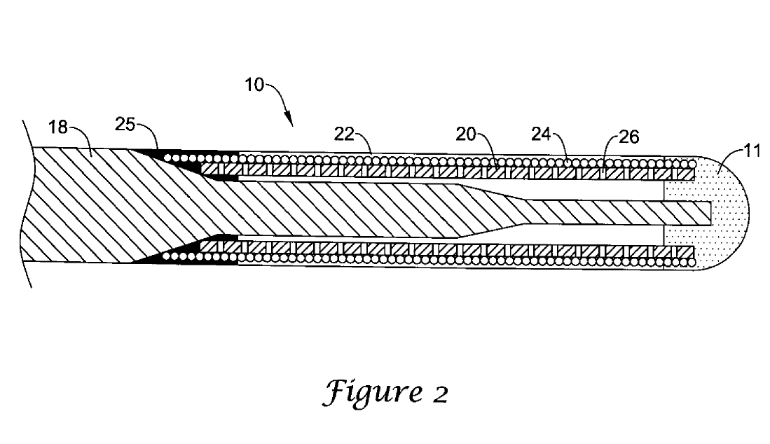

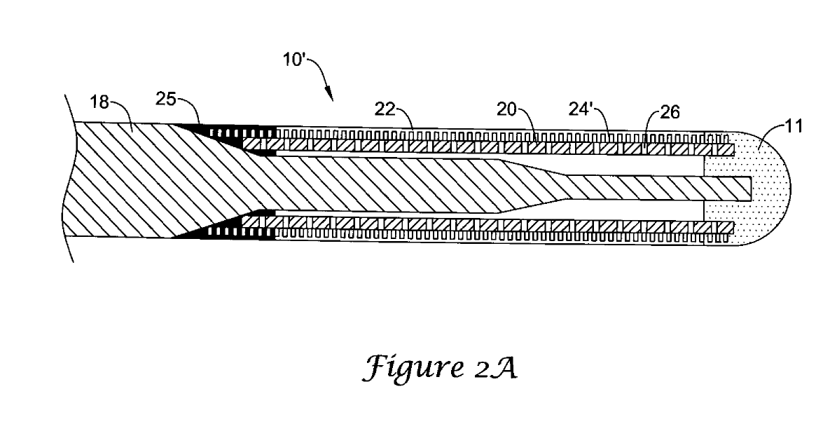

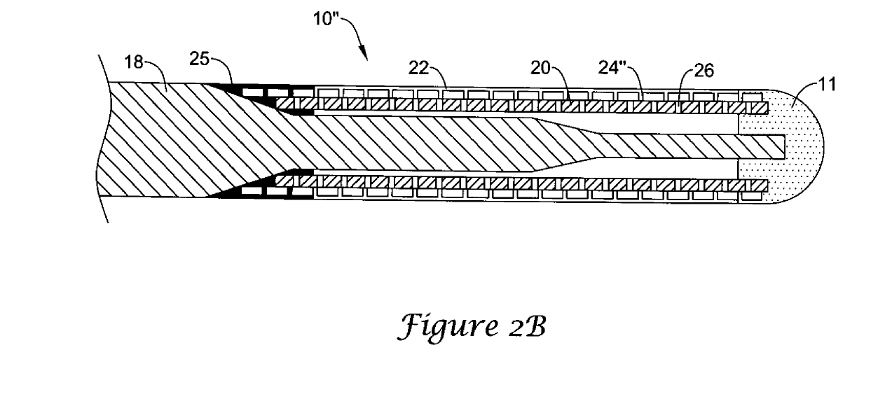

Turning now to FIG. 2, here it can be seen that an example guidewire 10 may include a shaft including a core wire 18, a tubular member 20 disposed over at least a portion of core wire 18, and a coil 24 disposed along at least a portion of the exterior surface of tubular member 20. A rounded or generally atraumatic distal tip 11, such as a can be formed at the distal end of guidewire 10. The distal tip 11 may be any or a broad variety of suitable structures, for example, a solder tip, a weld tip, a pre-made or pre-formed metallic or polymer structure, or the like, that is attached or joined to the distal end of the tubular member 20, core wire and/or the coil 24 using a suitable attachment technique.

The core wire 18 that may be attached to the tubular member 20, and extend from a location within the tubular member 20 and/or from the proximal end of the tubular member 20 to the proximal end of the guidewire 10. However, in other embodiments, the core member 18 may be absent, and/or the tubular member 20 may extend to the proximal end of the guidewire 10. For example, in some other embodiments, the tubular member 20 may extend along substantially the entire length of the guidewire 10, for example, form the proximal end to the distal end of the guidewire, and the core member 18 may be present and disposed within at least a portion of the tubular member 20, or may be absent, as desired. In some embodiments, core wire 18 may extend to the distal end of tubular member 20. In other embodiments, tubular member 20 may extend distally beyond the distal end of core wire 18. Additionally, the core wire 18 may extend to and/or into distal tip 11, or may end proximally thereof. In some embodiments, a shaping structure, such as a shaping ribbon, wire, or coil, may be attached to and extend distally beyond the distal end of core wire 18.

Tubular member 20 can be attached to core wire 18 in any suitable manner. For example, tubular member 20 and core wire 18 can be attached at the proximal end of tubular member 20, the distal end of tubular member 20, both, and/or at any suitable position therebetween. For example, tubular member 20 and core wire 18 can be attached at a bond point 25 as shown in FIG. 2. Bond point 25 may be an adhesive bond, a solder bond, a weld, a braze, a mechanical fit or bond, or the like, or others. Additionally, the distal end of the core wire 18 may be connected to the distal end of the tubular member 20 and/or the coil via the distal tip 11. Some additional description regarding the attachment of core wires and tubular members can be found in U.S. Patent Pub. No. 2004/0181174-A2, the entire contents of which are herein incorporated by reference.

In at least some embodiments, tubular member 20 includes a plurality of slots 26 formed therein. Slots 26 may be micromachined or otherwise created in tubular member 20, and may be configured to make tubular member 20 more flexible in bending. It is worth noting that, to the extent applicable, the methods for forming slots 26 and different configurations for slots can include, for example, any of the appropriate micromachining methods and other cutting methods and slot configurations disclosed in U.S. Pat. Publication Nos. US 2003/0069522; and US 2004/0181174-A2; and U.S. Pat. Nos. 6,766,720; and 6,579,246, the entire disclosures of which are herein incorporated by reference. These and other cutting methods may also include saw cutting (e.g., diamond grit embedded semiconductor dicing blade), etching (for example using the etching process described in U.S. Pat. No. 5,106,455, the entire disclosure of which is herein incorporated by reference), laser cutting, electrical discharge machining (and/or electron discharge machining), or the like. It should be noted that the methods for manufacturing guidewire 10 may include forming slots 26 in tubular member 20 using any of these or other manufacturing steps.

Various embodiments of arrangements and configurations of slots 26 are contemplated. Slots 26 may be generally arranged to be perpendicular to the longitudinal axis of tubular member 20. This arrangement can, alternatively, be described as having slots 26 lying within a plane that is normal to the longitudinal axis of tubular member 20. In other embodiments, slots 26 may be formed at an angle relative to a plane that is normal to the longitudinal axis. In some embodiments, slots 26 may be formed part way through tubular member 20, while in other embodiments, slots 26 may extend all the way through tubular member 20. Any one or more of the individual slots 26 may extend only partially around the longitudinal axis of tubular member 20. In yet other embodiments, slots 26 may extend in a helical arrangement about the longitudinal axis of tubular member 20. Slots 26 may be formed in groups of two, three, or more slots 26, which may be located at substantially the same location along the axis of tubular member 20, and may be substantially perpendicular to the longitudinal axis. Additionally, each of the groups of slots may be offset radially from adjacent groups of slots, for example, such that slots in adjacent groups do not necessarily align. Additionally, the density of slots along the length of the tubular member 20 may be constant, or may vary, for example, to achieve different flexibility characteristics as desired.

As indicated above, coil 24 may be disposed along the exterior surface of tubular member 20. In some embodiments, coil 24 may be disposed directly on the exterior surface of tubular member 20. Alternatively, a sleeve or jacket (not shown) may be disposed between tubular member 20 and coil 24. The sleeve or jacket may resemble sheath 22 discussed below, in form and/or material, or take any other suitable configuration. The exact position and/or configuration of coil 24 relative to tubular member 20 can also vary considerably. For example, in some embodiments coil 24 may extend from the proximal end to the distal end of tubular member 20. This may include the proximal and distal ends of both tubular member 20 and coil 24 axially aligning with one another. However, this need not be the case as the proximal end of coil 24 may be disposed distally of the proximal end of tubular member 20 and/or the distal end of coil 24 may be disposed proximally of the distal end of tubular member 20. Moreover, coil 24 may extend distally beyond the distal end of tubular member 20, proximally beyond the proximal end of tubular member 20, or both.

The coil 24 may be attached directly to the tubular member and/or to the core 18, or both, in any suitable manner. For example, tubular member 20 and coil 24 can be attached at the proximal end of tubular member 20, the distal end of tubular member 20, both, and/or at any suitable position therebetween. For example, tubular member 20 and coil 24 can be attached at bond point 25 as shown in FIG. 2. Similarly, the core 18 can be attached at the coil 24 at the proximal end of the coil 24, for example, at a bond point 25. Again, the bond point 25 may be an adhesive bond, a solder bond, a weld, a braze, a mechanical fit or bond, or the like, or others. Additionally, the distal end of the coil may be connected to the distal end of the tubular member 20 and/or the core 18 via the distal tip 11.

The coil 24 may be formed of round wire or flat ribbon ranging in dimensions to achieve the desired flexibility. It can also be appreciated that other cross-sectional shapes or combinations of shapes (e.g., oval, rectangular, square, triangle, polygonal, and the like, or any suitable shape) may be utilized without departing from the spirit of the invention. For example, FIG. 2A depicts guidewire 10′, which is otherwise similar to guidewire 10, where coil 24′ is a generally rectangular ribbon that is “edge-wound” about tubular member 20 (i.e., wound with the smaller edges of the rectangular ribbon disposed adjacent tubular member 20). Alternatively, FIG. 2B depicts guidewire 10″, which is otherwise similar to guidewire 10, which is wound about tubular member 20 with the larger edges of the rectangular ribbon adjacent tubular member 20. It can be appreciated that numerous other embodiments are contemplated that utilize wires or ribbons that have these or other cross-sectional shapes wound about tubular member 20 in any suitable manner or configuration.

The coil 24 can be wrapped in a helical fashion by conventional winding techniques. The pitch of adjacent turns of coil 24 may be tightly wrapped so that each turn touches the succeeding turn or the pitch may be set such that coil 24 is wrapped in an open fashion. In some embodiments, the coil can have a pitch of up to about 0.04 inches, in some embodiments a pitch of up to about 0.02 inches, and in some embodiments, a pitch in the range of about 0.001 to about 0.004 inches. The pitch can be constant throughout the length of the coil 24, or can vary, depending upon the desired characteristics, for example flexibility. These changes in coil pitch can be achieved during the initial winding of the wire, or can be achieved by manipulating the coil after winding or after attachment to the guidewire. For example, in some embodiments, after winding of the coil 24, a larger pitch can be achieved on the distal portion of the coil 24 by simply pulling the coil. Additionally, in some embodiments, portions or all of the coil 80 can include coil windings that are pre-tensioned or pre-loaded during wrapping, such that each adjacent coil winding is biased against the other adjacent coil windings to form a tight wrap. Such preloading could be imparted over portions of, or over the entire length of the coil 24. The diameter of the coil 24 is preferably sized to fit around the guidewire tubular member 20, and to give the desired characteristics.

Because coil 24 may be disposed along the exterior surface of tubular member 20, in some embodiments, the outer diameter of the tubular member 20 may be configured to have a somewhat decreased outer diameter relative to the proximal portion of the core member 18 such that a relatively constant outer diameter may be achieved along the length of the guidewire. The size of tubular member 20, thus, may be appropriate for adding coil 24 while still producing a guidewire with the desired outer diameter, for example, in the range of about 0.005 to about 0.20 inches or so.

A sheath or covering 22 may be disposed over portions or all of core wire 18, tubular member 20, and/or coil 24 that may define a generally smooth outer surface for guidewire 10. In other embodiments, however, such a sheath or covering 22 may be absent from a portion of all of guidewire 10, such that coil 24 and/or tubular member 20 and/or core wire 18 may form portions or all of the outer surface.

The addition of the coil 24 about the tubular member 20 may provide guidewire 10 with a number of desirable features and characteristics. For example, coil 24 may include a radiopaque material that allows guidewire 10 to be more easily fluoroscopically visualized. In addition, coil 24 may serve as a base or template for sheath 22 to be disposed on. Moreover, coil 24 may provide guidewire 10 with a desirable level of flexibility, for example, near tip 11. Because guidewire 10 also includes tubular member 20, which may provide a high level of torque transmission, guidewire 10 may have a desirable balance of flexibility and torque transmission.

Additionally, because guidewire 10 includes tubular member 20 (as well as a number of additional structural features), it may have some features and/or characteristics that overlap with spring tip guidewires in addition to a number of distinct features, such as torque transmission characteristics. Consequently, some clinicians may prefer guidewire 10 for certain interventions due to the features and characteristics that guidewire 10 provides. In order to make it easier for the clinician to identify, for example, the type of tip configuration found in guidewire 10 and/or to distinguish guidewire 10 from a polymer tip guidewire, guidewire 10 may also include a coil or coil member 24 disposed along at least a portion of the length of tubular member 20. Because coil 24 may be disposed along the exterior of guidewire 10, it may allow a clinician to more easily select guidewire 10 as being a guidewire best suited for a particular intervention.

The materials that can be used for the various components of guidewire 10 may include those commonly associated with medical devices. For example, core wire 18, tubular member 20, and/or coil 24 may be made from a metal, metal alloy, a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS; N06625 such as INCONEL®625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONELR®400, NICKELVAC®400, NICORROS®400, and the like), nickel-cobalt-chromium-molybdenum alloys (e.g., UNS: R30035 such as MP35-N® R and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

As alluded to above, within the family of commercially available nickel-titanium or nitinol alloys, is a category designated “linear elastic” which, although is similar in chemistry to conventional shape memory and superelastic varieties, exhibits distinct and useful mechanical properties. By skilled applications of cold work, directional stress, and beat treatment, the material is fabricated in such a way that it does not display a substantial “superelastic plateau” or “flag region” in its stress/strain curve. Instead, as recoverable strain increases, the stress continues to increase in an essentially linear relationship until plastic deformation begins. In some embodiments, the linear elastic nickel-titanium alloy is an alloy that does not show any martensite/austenite phase changes that are detectable by DSC and DMTA analysis over a large temperature range.

For example, in some embodiments, there are no martensite/austenite phase changes detectable by DSC and DMTA analysis in the range of about −60° C. to about 120° C. The mechanical bending properties of such material are therefore generally inert to the effect of temperature over this very broad range of temperature. In some particular embodiments, the mechanical properties of the alloy at ambient or room temperature are substantially the same as the mechanical properties at body temperature. In some embodiments, the use of the linear elastic nickel-titanium alloy allows the guidewire to exhibit superior “pushability” around tortuous anatomy. Accordingly, components of guidewire 10, such as core wire 18, tubular member 20, and/or coil 24 may include linear elastic nickel-titanium alloy.

In some embodiments, the linear elastic nickel-titanium alloy is in the range of about 50 to about 60 weight percent nickel, with the remainder being essentially titanium.

In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. In some other embodiments, a superelastic alloy, for example a superelastic nitinol can be used to achieve desired properties.

In at least some embodiments, portions or all of core wire 18, tubular member 20, and/or coil 24, or other components of the guidewire 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of guidewire 10 to achieve the same result.

In some embodiments, a degree of MRI compatibility is imparted into the guidewire 10. For example, to enhance compatibility with Magnetic Resonance Imaging (MRI) machines, it may be desirable to make core wire 18, tubular member 20, coil 24, and/or other portions of the medical device 10, in a manner that would impart a degree of MRI compatibility. For example, core wire 189, tubular member 20, and/or coil 24, or portions thereof, may be made of a material that does not substantially distort the image and create substantial artifacts (artifacts are gaps in the image). Certain ferromagnetic materials, for example, may not be suitable because they may create artifacts in an MRI image. Core wire 18, tubular member 20, and/or coil 24, or portions thereof, may also be made from a material that the MRI machine can image. Some materials that exhibit these characteristics include, for example, tungsten, Elgiloy, MP35N, nitinol, and the like, and others.

Referring now to core wire 18, the entire core wire 18 can be made of the same material along its length, or in some embodiments, can include portions or sections made of different materials. In some embodiments, the material used to construct core wire 18 is chosen to impart varying flexibility and stiffness characteristics to different portions of core wire 18. For example, the proximal region and the distal region of core wire 18 may be formed of different materials, for example materials having different moduli of elasticity, resulting in a difference in flexibility. In some embodiments, the material used to construct the proximal region can be relatively stiff for pushability and torqueability, and the material used to construct the distal region can be relatively flexible by comparison for better lateral trackability and steerability. For example, the proximal region can be formed of straightened 304v stainless steel wire or ribbon and the distal region can be formed of a straightened super elastic or linear elastic alloy, for example a nickel-titanium alloy wire or ribbon.

In embodiments where different portions of core wire 18 are made of different materials, the different portions can be connected using any suitable connecting techniques. For example, the different portions of core wire 18 can be connected using welding (including laser welding), soldering, brazing, adhesive, or the like, or combinations thereof. Additionally, some embodiments can include one or more mechanical connectors or connector assemblies to connect the different portions of core wire 18 that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. No. 09/972,276 filed on Oct. 5, 2001, Ser. No. 10/068,992 filed on Feb. 28, 2002, and Ser. No. 10/375,766 filed on Feb. 26, 2003, which are incorporated herein by reference.

Core wire 18 can have a solid cross-section, but in some embodiments, can have a hollow cross-section. In yet other embodiments, core wire 18 can include a combination of areas having solid cross-sections and hollow cross sections. Moreover, core wire 18, or portions thereof, can be made of rounded wire, flattened ribbon, or other such structures having various cross-sectional geometries. The cross-sectional geometries along the length of core wire 18 can also be constant or can vary. For example, FIG. 2 depicts core wire 18 as having a round cross-sectional shape. It can be appreciated that other cross-sectional shapes or combinations of shapes may be utilized without departing from the spirit of the invention. For example, the cross-sectional shape of core wire 18 may be oval, rectangular, square, polygonal, and the like, or any suitable shape.

Sheath 22 may be made from a polymer or any other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like. In some embodiments sheath 22 can be blended with a liquid crystal polymer (LCP). For example, the mixture can contain up to about 6% LCP. This has been found to enhance torqueability. By employing selection of materials and processing techniques, thermoplastic, solvent soluble, and thermosetting variants of these and other materials can be employed to achieve the desired results.

In some embodiments, the exterior surface of the guidewire 10 (including, for example, the exterior surface of core wire 18, tubular member 20 and/or coil 24) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of sheath 22, or in embodiments without a sheath 22 over portion of core wire 18 and/or tubular member, or other portions of device 10. Alternatively, sheath 22 may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath 22 may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

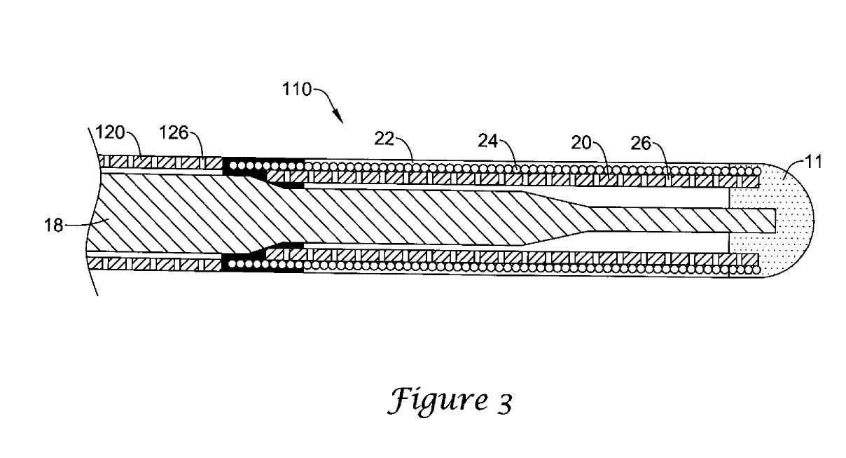

In at least some embodiments, it may be desirable to add one or more addition tubular members including one or more with an outer diameter that is larger that tubular member 20. For example, FIG. 3 illustrates another example guidewire 110 that is similar to guidewire 10 except that a second tubular member 120 having a larger outer diameter that tubular member 20 is disposed over core wire 18. In some embodiments, tubular member 120 can be positioned proximally of tubular member 20 and/or coil 24, or may include a portion that may overlap with the tubular member 20 and/or coil 24. However, the exact positioning of second tubular member 120 relative to tubular member 20 and/or coil 24 may include essentially any suitable position including second tubular member 120 being axially aligned with tubular member 20 and/or coil 24 or having a distal end that is disposed distal of the proximal end of tubular member 20 and/or coil 24. The second tubular member 120 may include structure and materials similar to those discussed above regarding the tubular member 20.

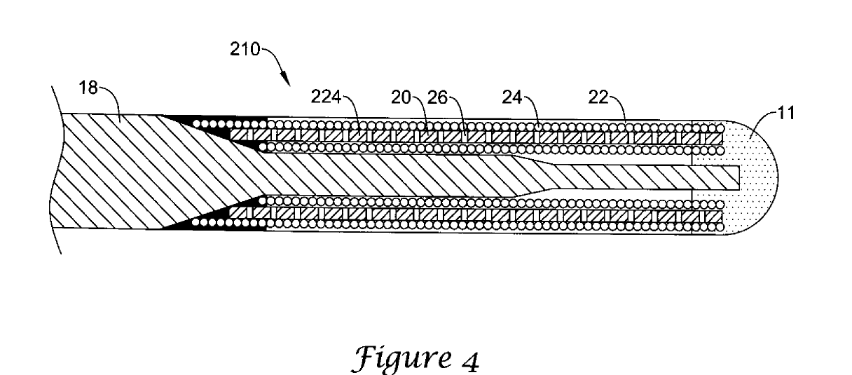

Another example guidewire 210 is depicted in FIG. 4. Guidewire 210 is similar to guidewire 10 except that guidewire 210 includes a second coil 224, for example, positioned between tubular member 20 and core wire 18. Coil 224 may include structure and materials similar to those discussed above regarding the coil 24. In some embodiments, the coil 224 may be made from a radiopaque material, and may function as a marker member. In addition, coil 224 may fulfill other functions such as partially or wholly filling the axial space between tubular member 20 and core wire 18. This may, for example, aid in center core wire 18 within tubular member 20.

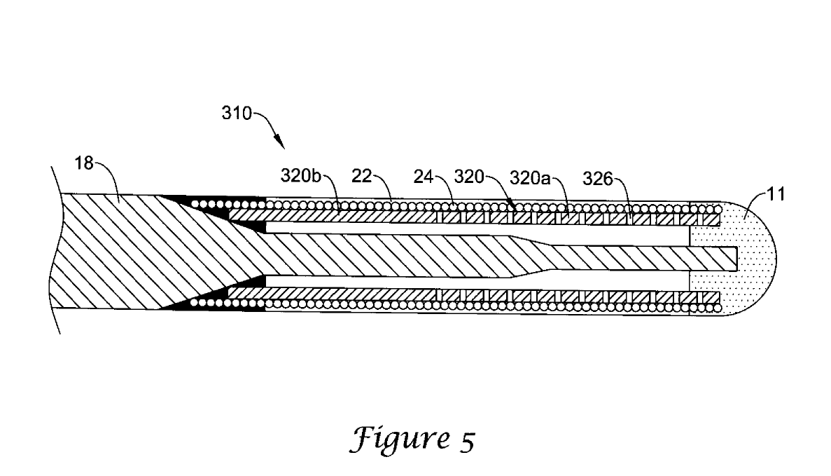

FIG. 5 illustrates another example guidewire 310. Guidewire 310 is similar to other guidewires disclosed herein except that tubular member 320 includes a first section 320a having slots 326 formed therein and a second section 320b free from slots. In some embodiments, sections 320a/320b are discrete tubular members that may or may not be attached to one another using any suitable joining technique such as any of those discussed herein. Alternatively, sections 320a/320b may be defined by simply disposing slots 326 along a portion of tubular member 320 so as to defined slotted section 320a.

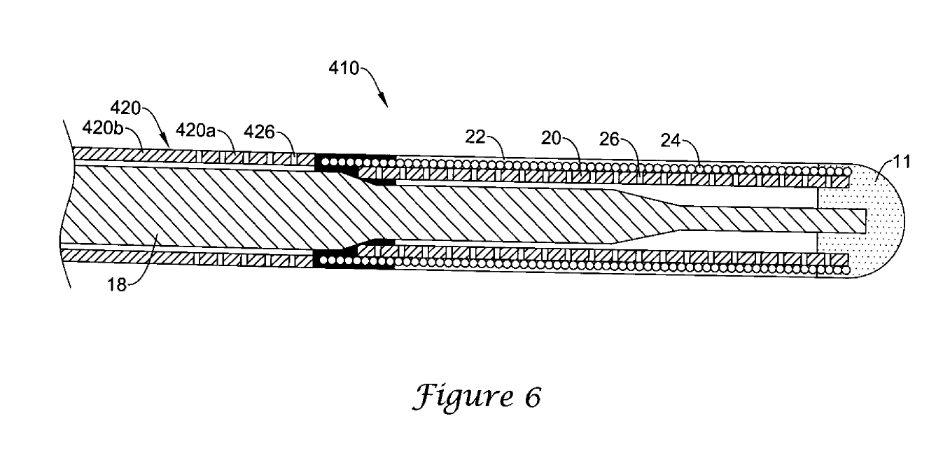

Another example guidewire 410 is depicted in FIG. 6. Guidewire 410 is similar to other guidewires disclosed herein except that guidewire 410 includes a second tubular member 420 having a first section 420a having slots 426 formed therein and a second section 420b free from slots. Just like in tubular member 320, sections 420a/420b of tubular member 420 can be formed from separate tubular members that may or may not be attached to one another or a singular tubular member 420 with slots 426 formed in only a portion thereof to define slotted sections 420a.

It should be noted that numerous guidewires are contemplated that combine the features of the various guidewires disclosed herein. For example, guidewires are contemplated that include both a first tubular member (e.g., 20/320), a second tubular member (e.g., 120/420), and a second coil (e.g., coil 224). In these embodiments, at least some include tubular members having slotted sections of first tubular member (e.g., 20/320), second tubular member (e.g., 120/420), or both.

It should be understood that this disclosure is, in many respects, only illustrative. Changes may be made in details, particularly in matters of shape, size, and arrangement of steps without exceeding the scope of the invention. In addition, many of the structures, material, or methods or combinations thereof described or shown in one or more embodiments may be incorporated into other embodiments as desired. The invention's scope is, of course, defined in the language in which the appended claims are expressed.

1. A guidewire, comprising:
an elongate shaft having a proximal region and a distal region;
a metallic tubular member disposed over the distal region of the shaft, the tubular member having an outer surface;
wherein a plurality of slots are defined in the tubular member; and
a coil disposed along the outer surface of the tubular member.

2. The medical device of claim 1, wherein the tubular member includes a first section and a second section, and wherein the slots are defined in the first section and the second section is free of slots.

3. The medical device of claim 1, further comprising a second coil disposed within the tubular member.

4. The medical device of claim 3, wherein the second coil includes a radiopaque material.

5. The medical device of claim 3, wherein a space is defined between the tubular member and the shaft, and wherein the second coil disposed within the space.

6. The medical device of claim 5, wherein the second coil substantially fills the space.

7. The medical device of claim 1, further comprising a second tubular member disposed over the shaft adjacent the tubular member.

8. The medical device of claim 7, wherein a plurality of slots are defined in the second tubular member.

9. The medical device of claim 8, wherein the second tubular member includes a first section and a second section, and wherein the slots are defined in the first section and the second section is free of slots.

10. The medical device of claim 7, wherein the second tubular member includes a distal end that is disposed proximally of a proximal end of the tubular member.

11. The medical device of claim 7, wherein the second tubular member includes a distal end that is disposed distally of a proximal end of the tubular member.

12. The medical device of claim 7, wherein the second tubular member includes a distal end that is axially aligned with a proximal end of the tubular member.

13. The medical device of claim 7, wherein the tubular member has a first outer diameter and the second tubular member has a second outer diameter that is larger than the first outer diameter.

14. A guidewire, comprising:
an elongate core wire having a proximal region and a distal region;
a metallic tubular member disposed over the distal region of the core wire, the tubular member having an outer surface;
wherein the tubular member includes a plurality of slots therein; and
a coil disposed along the outer surface of the tubular member.

15. The guidewire of claim 14, wherein the tubular member includes a slotted region having the slots defined therein, and the tubular member further includes a non-slotted region adjacent to the slotted region.

16. The guidewire of claim 15, wherein the slotted region is disposed distally of the non-slotted region.

17. The guidewire of claim 14, further comprising a second coil disposed within the tubular member.

18. The guidewire of claim 14, further comprising a second tubular member disposed adjacent the tubular member.

19. The medical device of claim 18, wherein the second tubular member includes a slotted region having a plurality of slots defined therein.

20. The medical device of claim 19, wherein a non-slotted region is defined in the second tubular member adjacent to the slotted region.

21. The medical device of claim 20, wherein the tubular member has a first outer diameter and the second tubular member has a second outer diameter that is larger than the first outer diameter.

22. A guidewire, comprising:
an elongate shaft having a proximal region and a distal region, the distal region including a metallic tubular member and a coil disposed over the tubular member, wherein the tubular member includes at least a region having a plurality of slots formed therein.

23. A method for manufacturing a medical device, the method comprising:
providing an elongate shaft having a proximal region and a distal region;
disposing a metallic tubular member over the distal region of the shaft, the first tubular member having an outer surface and having at least a region with a plurality of slots formed therein; and
disposing a coil along the outer surface of the tubular member.
